(19) United States
(12) Patent Application Publication
(10) Pub. No.: US 2020/0306474 A1
(43) Pub. Date: Oct. 1, 2020

(54) INFLATABLE SOFT PALATE SCAFFOLDING DEVICE

(71) Applicant: FORAMIS MEDICAL GROUP INC., Auburndale, FL (US)

(72) Inventors: J. Christopher Flaherty, Auburndale, FL (US); Tai Kien, Groton, MA (US); John N. Irwin, III, Stamford, CT (US); R. Maxwell Flaherty, Auburndale, FL (US)

(21) Appl. No.: 16/652,116

(22) PCT Filed: Oct. 4, 2018
(86) PCT No.: PCT/US2018/054351

§ 371 Date: Mar. 30, 2020

Related U.S. Application Data
(60) Provisional application No. 62/567,943, filed on Oct. 4, 2017.

(51) Int. Cl. A61M 16/04

(52) U.S. Cl. CPC A61M 16/0445; A61M 16/045; A61M 2205/0222; A61M 2205/0238; A61M 2210/065; A61M 2210/1028; A61M 2210/1032; A61M 2205/273; A61M 2210/0618

(57) ABSTRACT

A medical device for the treatment of one or more sleep apnea disorders such as obstructive sleep apnea in a patient is provided. The device includes a scaffolding element positioned about a distal portion of an elongate shaft. The scaffolding element receives a volume of fluid to expand the scaffolding element to exert a force upon a soft palate and/or base of a tongue of a patient. The scaffolding element provides a helical breathing pathway when inserted into an airway of the patient and transitioned to an expanded state.

### RELATED APPLICATIONS

This application claims the benefit of U.S. Provisional Application Ser. No. 62/567,943, titled “Inflatable Soft Palate Scaffolding Device”, filed Oct. 4, 2017, the content of which is incorporated herein by reference in its entirety for all purposes.

This application is related to:
U.S. patent application Ser. No. 13/120,964, filed Mar. 25, 2011, issued as U.S. Pat. No. 10,022,262;
U.S. patent application Ser. No. 16/007,549, filed Jun. 13, 2018, published as United States Publication Ser. No. ______;
U.S. patent application Ser. No. 13/130,869, filed May 24, 2011, issued as U.S. Pat. No. 9,132,028;
U.S. patent application Ser. No. 14/818,914, filed Aug. 5, 2015, published as United States Publication No. 2015-0342779;
U.S. patent application Ser. No. 13/985,977, filed Aug. 16, 2013, issued as U.S. Pat. No. 9,668,911;
U.S. patent application Ser. No. 15/611,085, filed Jun. 1, 2017, published as United States Publication No. 2017-0266034;
   the content of each of which is incorporated herein by reference in its entirety for all purposes.

### FIELD

The present inventive concepts relate generally to systems, devices, and methods for treatment of sleep apnea.

### BACKGROUND

Sleep apnea syndrome, and in particular obstructive sleep apnea, afflicts an estimated 2-5% of the general population and is due to episodic upper airway obstruction during sleep. Those afflicted with obstructive sleep apnea experience sleep fragmentation and intermittent, complete or nearly complete cessation of ventilation during sleep with potentially severe degrees of oxyhemoglobin unsaturation. These issues can be translated clinically into debilitating daytime sleepiness, cardiac dysrhythmias, pulmonary-artery hypertension, congestive heart failure and cognitive dysfunction. Other problems related to sleep apnea include carbon dioxide retention during wakefulness as well as during sleep, and continuous reduced arterial oxygen tension. Hypersomnolent sleep apnea patients may be at risk for excessive mortality from these factors as well as from an elevated risk for accidents such as while driving or operating other potentially dangerous equipment.

Obstructive sleep apnea occurs due to a collapse of soft tissue within the upper airway during sleep. Apnea is the term for suspension of breathing. During an apnea event (an “apnea”), there is no movement of the muscles of respiration. The ongoing force of inspiration serves to generate increasingly negative pressure within the pharynx, causing further collapse. The lack of respiration results in inadequate blood oxygenation, and rising carbon dioxide levels. The cardiovascular response produces an increase in the blood pressure and pulse. Cardiac arrhythmia's can occur. The carbon dioxide increase and oxygen desaturation triggers a transition to a lighter sleep stage, usually without wakefulness. This transition brings a return to tonicity of the muscles of the upper airway, allowing normal breathing to resume. The person then returns to deeper stages of sleep and the process is repeated. The disease is quantified in terms of respiratory disturbances per hour. Mild disease begins at 2-3 APNEAS per hour, and it is not uncommon to find patients with indices of 75 or more.

Not surprisingly, sleep is extremely fragmented and of poor quality in persons suffering from sleep apnea. As a result, such persons typically feel tired upon wakening and may fall asleep at inappropriate times during the day. All aspects of quality of life, from physical and emotional health, to social functioning are impaired by obstructive sleep apnea.

The treatment of sleep apnea has included such surgical interventions as Uvulopalatopharyngoplasty (UPPP) gastric surgery for obesity, and maxillo-facial reconstruction. Another mode of surgical intervention used in the treatment of sleep apnea is tracheostomy. These treatments constitute major undertakings with considerable risk of post-operative mortality. In UPPP, any remaining tonsil tissue and a portion of soft palate is removed. The procedure often increases the nasopharyngeal airway. However, UPPP does not always fix a sagging soft palate nor does it address apnea caused by obstructions caused by the base of the tongue being deeper in the oropharynx part of the airway. These surgical techniques are extremely invasive, requiring general anesthesia, and a prolonged, painful recovery.

LAUP, or Laser-Assisted Uvulopalatoplasty, is a modification of the above-mentioned technique, but has had mixed success and cannot solve obstructions behind the base of the tongue.

Radio frequency tissue ablation (RFTA) with the trade name “Somnoplasty”, has been used to shrink the soft palate, uvula and reduce tongue volume in the treatment of snoring and obstructive sleep apnea. Somnoplasty utilizes a radiofrequency tool that generates heat to create coagulative lesions at specific locations within the upper airway. The lesions created by the procedure are naturally resorbed in approximately three to eight weeks, reducing excess tissue volume and increasing the airway opening. More than one session is typically required, and other surgeries may still be necessary in moderate to severe cases, and there are occasional problems with morbidity.

Another area of surgical interest lies in techniques designed to pull the tongue anteriorly. The most recent such surgical system designed to treat snoring (as well as obstructive sleep apnea) was approved by the FDA in February 1998. Known as the tongue suspension procedure (with the trade name Repose), it is intended to pull the tongue forward, thereby keeping the tongue from falling into the airway during sleep. The system utilizes a bone screw inserted into the mandible. The screw attaches to a non-absorbable suture which travels the length of the tongue and back. Similarly, the hyoid bone can be drawn anteriorly with two distinct screws, also attached to the mandible.

These conventional treatments continue to suffer poor or partial cure rates. The failures lie in their inability to maintain patency in the retropalatal region and retroglossal region (the caudal margin of the soft palate to the base of the epiglottis). The poor success rates combined with high morbidity from some of the surgical interventions, contribute to an ongoing need for more effective treatments for sleep apnea and/or snoring.

Pharmacological therapy aimed at stimulating upper airway muscle to reduce apneas also have, in general, been disappointing. In addition, side effects from the pharmacological agents that have been used are frequent. Thus, medical practitioners continue to seek non-invasive modes of treatment for sleep apnea with high success rates and high patient compliance including, for example in cases of minor to moderate sleep apnea relating to obesity, weight loss through a regimen of exercise and regulated diet.

Other non-surgical treatments for sleep apnea include the use of oral devices and appliances that work to prevent the tongue from falling backwards or help reduce the collapse of the soft palate. These involve the use of retainers that push the lower jaw forward, thereby pulling the tongue slightly forward and, in some cases, helping elevate the soft palate. Also, there are devices that pull on the tongue to keep it forward during sleep. These current oral devices, typically do not create a significant improvement except in mild to moderate cases and can be associated with movement of the teeth over time of problems with the temporomandibular joint.

Recent work in the treatment of sleep apnea has included the use of continuous positive airway pressure (CPAP) to maintain the airway of the patient in a continuously open state during sleep. CPAP, by delivering a stream of air under pressure through the nose or mouth, stents the airway (keeping it open) so that apneas are reduced and breathing during sleep becomes unobstructed.

Although CPAP has been found to be very effective and well accepted, it suffers from some of the same limitations, although to a lesser degree, as do the surgical options; specifically, a significant proportion of sleep apnea patients do not tolerate CPAP well. Thus, development of other viable non-invasive therapies has been a continuing objective in the art.

While the above-identified conventional devices and surgical techniques are purported to treat upper airway instability, such as OSA or snoring, they are successful, if at all, in only a limited pool of patients or under limited circumstances. While CPAP therapy has had significant success in reducing or eliminating apneas through the delivery of air under pressure, CPAP treatment suffers from patient non-compliance and cannot be tolerated by an ample minority of patients. Therefore, there remains a relatively large number of patients whose airway disorder is believed to be treatable using an intraoral appliance, yet conventional appliances are ineffective, overly burdensome, uncomfortable, or any combination thereof.

There is a need for improved systems, devices, and methods for the treatment of sleep apnea, and in particular, sleep apnea caused by an obstruction of the airway by soft palate tissue.

### SUMMARY

According to an aspect of the present inventive concepts, an airway scaffolding device for a patient comprises: an elongate shaft with a distal portion; a scaffolding element positioned about the distal portion of the elongate shaft, the scaffolding element configured to receive a volume of fluid to expand the scaffolding element to exert a force upon a soft palate and/or base of a tongue of the patient; a valve assembly positioned proximal to the scaffolding element; and a lumen disposed between the valve assembly and the scaffolding element. The fluid is introduced through the valve assembly and travels through the lumen into the scaffolding element. The scaffolding element provides a helical breathing pathway when inserted into an airway of the patient and transitioned to an expanded state via the fluid introduced through the valve assembly. The valve assembly is configured to reduce egress of fluid from the scaffolding element to maintain the scaffolding element in the expanded state.

In some embodiments, the device comprises a single-use device.

In some embodiments, the device comprises a multiple-use device. The device can be configured to be cleaned between uses.

In some embodiments, the scaffolding element is configured to be expanded after insertion into the patient's airway.

In some embodiments, the scaffolding element comprises a balloon.

In some embodiments, the scaffolding element is configured to expand into a helical geometry.

In some embodiments, the scaffolding element is configured to be deflated prior to removal from the patient.

In some embodiments, the scaffolding element is configured to be removed from the patient without deflation.

In some embodiments, the scaffolding element is configured to be filled with a liquid.

In some embodiments, the scaffolding element is configured to be filled with a gas. The gas can comprise air.

In some embodiments, the scaffolding element comprises a compliant material. The scaffolding element can further comprise a non-compliant material.

In some embodiments, the scaffolding element comprises a non-compliant material.

In some embodiments, the scaffolding element comprises a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations thereof.

In some embodiments, the scaffolding element comprises a length less than or equal to 4 cm. The scaffolding element can comprise a length less than or equal to 7 cm, and/or less than or equal to 10 cm.

In some embodiments, the scaffolding element comprises a length between 1 cm and 4 cm. The scaffolding element can comprise a length between 1.5 cm and 2.8 cm.

In some embodiments, the scaffolding element comprises a thickness of at least 0.0001″. The scaffolding element can comprise a thickness of at least 0.001″ and/or at least 0.010″.

In some embodiments, the shaft comprises a distal end, the scaffolding element comprises a distal end, and the distal end of the scaffolding element is positioned within at least 10 mm of the distal end of the shaft. The distal end of the scaffolding element can be positioned within at least 5 mm of the distal end of the shaft.

In some embodiments, the scaffolding element comprises a spiral major diameter of between 0.5 cm and 5.0 cm when expanded. The scaffolding element can comprise a spiral major diameter of between 1.5 cm and 2.5 cm when expanded. The scaffolding element can comprise a spiral major diameter of between 1.7 cm and 2.0 cm when expanded.

In some embodiments, the scaffolding element comprises a burst pressure of at least 5 psi. The scaffolding element can comprise a burst pressure of at least 10 psi, at least 30 psi, and/or at least 50 psi.

In some embodiments, the scaffolding element comprises a fill volume of less than or equal to 30 ml. The scaffolding element can comprise a fill volume of less than or equal to 20 ml.

In some embodiments, the scaffolding element comprises a pitch of greater than or equal to 2 mm. The scaffolding element can comprise a pitch of greater than or equal to 4 mm, and/or greater than or equal to 6 mm.

In some embodiments, the scaffolding element comprises a pitch less than or equal to 20 mm. The scaffolding element can comprise a pitch less than or equal to 15 mm, and/or less than or equal to 10 mm.

In some embodiments, the scaffolding element comprises a tube wrapped around the shaft. The tube can be adhesively attached to the shaft. The tube can be adhesively attached to the shaft at multiple discrete locations. The scaffolding element can comprise a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations thereof.

In some embodiments, the scaffolding element comprises a tube that circumferentially surrounds the shaft. The tube can comprise a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations thereof. The scaffolding device can further comprise adhesive positioned between the tube and the shaft. The tube can comprise a proximal portion and a distal portion, and the adhesive can be circumferentially positioned at the tube proximal portion and distal portion. The tube can be positioned in a twisted arrangement. The twisted arrangement can comprise the tube being twisted between 10° and 2160° about the shaft. The twisted arrangement can comprise the tube being twisted between 90° and 1080° about the shaft. The twisted arrangement can comprise the tube being twisted between 360° and 1080° about the shaft. The tube can comprise a resiliently biased ID, and the shaft can comprise a resiliently biased OD, and the resiliently biased ID of the tube can be less than the resiliently biased OD of the shaft. The resiliently biased ID of the tube can be at least 30% smaller than the resiliently biased OD of the shaft. The resiliently biased ID of the tube can be at least 40%, at least 50%, at least 60%, and/or at least 70% smaller than the resiliently biased OD of the shaft. The adhesive can comprise UV curable adhesive. The adhesive can be applied in a helical pattern such that the tube inflates in a helical geometry. The adhesive can be cured in a helical pattern such that the tube inflates in a helical geometry.

In some embodiments, the helical breathing pathway comprises a pathway with a cross sectional area that is at least 10% of the area defined by the major diameter of the scaffolding element when the scaffolding element is expanded. The helical breathing pathway can comprise a pathway with a cross sectional area that can be at least 20%, at least 35%, and/or at least 45% of the area defined by the major diameter of the scaffolding element when the scaffolding element is expanded.

In some embodiments, the helical breathing pathway comprises a cross sectional area of at least 0.314 cm². The helical breathing pathway can comprise a cross sectional area of at least 0.628 cm², at least 1.099 cm², and/or at least 1.413 cm².

In some embodiments, the valve is positioned on the proximal end of the shaft.

In some embodiments, the valve is positioned within the lumen.

In some embodiments, the valve comprises a one-way valve.

In some embodiments, the valve comprises a valve selected from the group consisting of: duck-bill valve; slit valve; spring-activated valve; electronically actuatable valve; one-way valve; and combinations thereof.

In some embodiments, the valve is configured to be opened to allow fluid to exit the scaffolding element. The valve can be configured to be opened by compression of the valve.

In some embodiments, the valve is configured to be removed to allow fluid to exit the scaffolding element.

In some embodiments, the valve is configured to maintain a pressure within the scaffolding element of at least 5 psi. The valve can be configured to maintain a pressure within the scaffolding element of at least 10 psi, 30 psi, and/or 50 psi.

In some embodiments, the valve comprises a pressure-relief valve.

In some embodiments, the valve comprises a cracking pressure of at least 0.1 psi. The valve can comprise a cracking pressure of at least 0.5 psi.

In some embodiments, the valve comprises a cracking pressure of less than or equal to 200 psi. The valve can comprise a cracking pressure of less than or equal to 50 psi.

In some embodiments, the valve comprises a nasal dilator.

In some embodiments, the valve comprises a first projection and a second projection, and the first and second projections are resiliently biased in a closed condition that obstructs fluid flow through the valve, and compression of the valve transitions the valve into an open condition.

In some embodiments, the shaft comprises a sealed distal end.

In some embodiments, the shaft comprises a rounded distal end.

In some embodiments, the shaft comprises a hole that provides a fluid connection between the lumen and the scaffolding element.

In some embodiments, the shaft comprises a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations thereof.

In some embodiments, the shaft comprises a material selected from the group consisting of: stainless steel; nitinol; and combinations thereof. The shaft can comprise a coiled configuration.

In some embodiments, the shaft comprises a diameter between 0.018″ and 0.300″. The shaft can comprise a diameter between 0.030″ and 0.120″.

In some embodiments, the shaft comprises a length of at least 4 cm. The shaft can comprise a length of at least 6 cm.

In some embodiments, the shaft comprises a length between 6 cm and 30 cm. The shaft can comprise a length between 8 cm and 20 cm, and/or between 10 cm and 13 cm.

In some embodiments, the lumen comprises a diameter of at least 0.005″. The lumen can comprise a diameter of at least 0.015″.

In some embodiments, the scaffolding device further comprises a nasal dilator.

In some embodiments, the scaffolding device further comprises a coating positioned on the shaft and/or scaffolding element. The coating can be positioned on the shaft and the scaffolding element. The coating can comprise a lubricious coating. The coating can comprise a coating selected from the group consisting of: a hydrophilic coating and/or material; a hydrophobic coating and/or material; a lubricant coating; a silicone lubricant; polytetrafluoroethylene (PTFE) coating and/or material; and combinations thereof. The coating can comprise a coating selected from the group consisting of: a lubricous coating; an antibiotic; an antihistamine; an analgesic; and combinations thereof.

In some embodiments, the scaffolding device further comprises a fluid delivery assembly including a reservoir for surrounding the fluid to be delivered to the scaffolding element. The fluid delivery assembly can fluidly attach to the valve and/or the lumen. The fluid delivery assembly can comprise threads configured to provide the attachment. The reservoir can comprise a syringe. The reservoir can comprise a flexible pouch. The pouch can comprise ribs that can be configured to maintain the pouch in an expanded state.

In some embodiments, the scaffolding device further comprises a filament configured to be inserted into the device to stiffen the device during insertion into the patient's airway. The scaffolding element can be configured to be inserted into the lumen.

In some embodiments, the scaffolding element comprises multiple peaks when expanded, and the scaffolding device further comprises one or more filaments, each filament fixedly attached to the multiple peaks of the scaffolding element. The one or more filaments can be configured to reduce deflection of the multiple peaks. The one or more filaments can be configured to maximize the volume of the helical breathing pathway. The one or more filaments can be configured to minimize restrictions of the helical breathing pathway.

According to another aspect of the present inventive concepts, an airway scaffolding device for a patient comprises: an elongate shaft with a distal portion and multiple slits positioned in the distal portion; a scaffolding element configured to receive a volume of fluid to expand the scaffolding element to exert a force upon a soft palate and/or base of a tongue of the patient; a valve assembly positioned proximal to the scaffolding element; and a lumen disposed between the valve assembly and the scaffolding element. The fluid is introduced through the valve assembly and travels through the lumen into the scaffolding element. The scaffolding element comprises multiple lobes positioned within the lumen. Each of the lobes is configured to pass thru a corresponding slit of the shaft as the scaffolding element is expanded. The lobes of the scaffolding element provide a breathing pathway when inserted into an airway of the patient and transitioned to an expanded state via the fluid introduced through the valve assembly. The valve assembly is configured to reduce egress of fluid from the scaffolding element to maintain the scaffolding element in the expanded state. The multiple lobes can comprise at least three lobes, and the multiple slits can comprise at least three slits.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a cross sectional view of a patient's oral cavity, consistent with the present inventive concepts.

FIG. 2 illustrates a side view of an airway scaffolding device, consistent with the present inventive concepts.

FIGS. 2A and 2B illustrate sectional and side sectional views of an airway scaffolding device, respectively, consistent with the present inventive concepts.

FIGS. 3A and 3B illustrate side views of an airway scaffolding device, in unexpanded and expanded conditions, respectively, consistent with the present inventive concepts.

FIG. 4 illustrates a kit for constructing an airway scaffolding device, consistent with the present inventive concepts.

FIGS. 4A-4D illustrate the steps for constructing the airway scaffolding device of FIG. 4, consistent with the present inventive concepts.

FIG. 5 illustrates a kit for constructing an airway scaffolding device, consistent with the present inventive concepts.

FIGS. 5A-5C illustrate the steps for constructing the airway scaffolding device of FIG. 5, consistent with the present inventive concepts.

FIGS. 6A and 6B illustrate cross sectional and side views of the distal portion of an unexpanded balloon in a distal portion of an airway scaffolding device, respectively, consistent with the present inventive concepts.

FIGS. 6C and 6D illustrate cross sectional and side views of the distal portion of an expanded balloon in a distal portion of an airway scaffolding device, respectively, consistent with the present inventive concepts.

FIGS. 7A and 7B illustrate side sectional views of the proximal portion of an airway scaffolding device comprising a valve having a nosepiece, consistent with the present inventive concepts.

FIGS. 8A-8D illustrate a method for inserting an airway scaffolding device into a patient's airway, consistent with the present inventive concepts.

FIG. 9 illustrates a side view of an airway scaffolding device, in an expanded condition, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

It is appreciated that certain features of the inventive concepts, which are, for clarity, described in the context of separate embodiments, may also be provided in combination in a single embodiment. Conversely, various features which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the disclosure have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Provided herein are airway scaffolding devices for a patient, such as a patient that has issues or potential issues with sleep apnea and/or snoring. The scaffolding devices of the present inventive concepts include a balloon or other scaffolding element that can be inflated or otherwise expanded with a fluid or other flowable material (“fluid” herein) to assume a spiral or otherwise helical geometry (“helical” or “spiral” herein). This helical geometry includes “peaks” and “valleys”. The peaks of the helical geometry can be used to apply a force to tissue of the patient's airway, such as soft palate tissue or tissue of the tongue that otherwise would restrict the breathing of the patient (e.g. tissue that collapses into the airway during sleep). When inserted into an airway of the patient, the valleys of the helical geometry provide a helical shaped lumen, a “helical breathing pathway”, that supports normal, unobstructed breathing (e.g. maintains an open lumen through the airway proximate the patient's soft palate and/or base of tongue during sleep). The scaffolding devices of the present inventive concepts can be “single use” (e.g. disposed of after a single night's sleep), or “multiple use” (e.g. used for a first night's sleep, and then again one or more additional night's sleep, such as when a cleaning procedure is performed prior to repeated use).

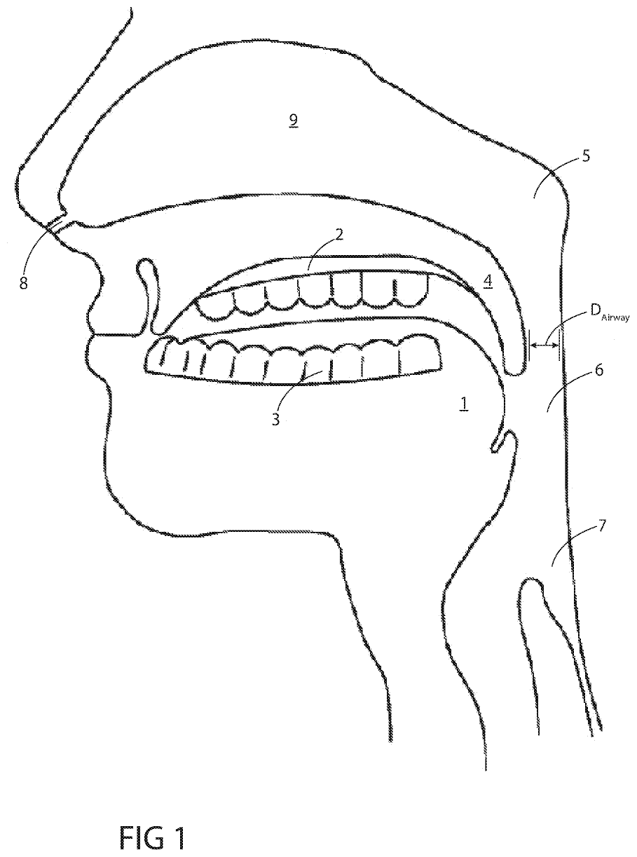

Referring to FIG. 1, a cross section of a patient's oral cavity is illustrated. The patient's oral cavity includes a tongue 1, an upper jaw 2, a lower jaw 3, a soft palate 4, and an epiglottis, as well as a nasopharynx region 5, an oropharynx region 6, and a laryngopharynx region 7. In some embodiments, an airway diameter, D_{Airway}, can comprise the distance between a posterior surface of soft palate 4 and a posterior surface of nasopharynx region 5. In addition, the patient's nose includes nasal valve 8, and a nasal passageway 9 that fluidly connects nasal valve 8 with nasopharynx region 5.

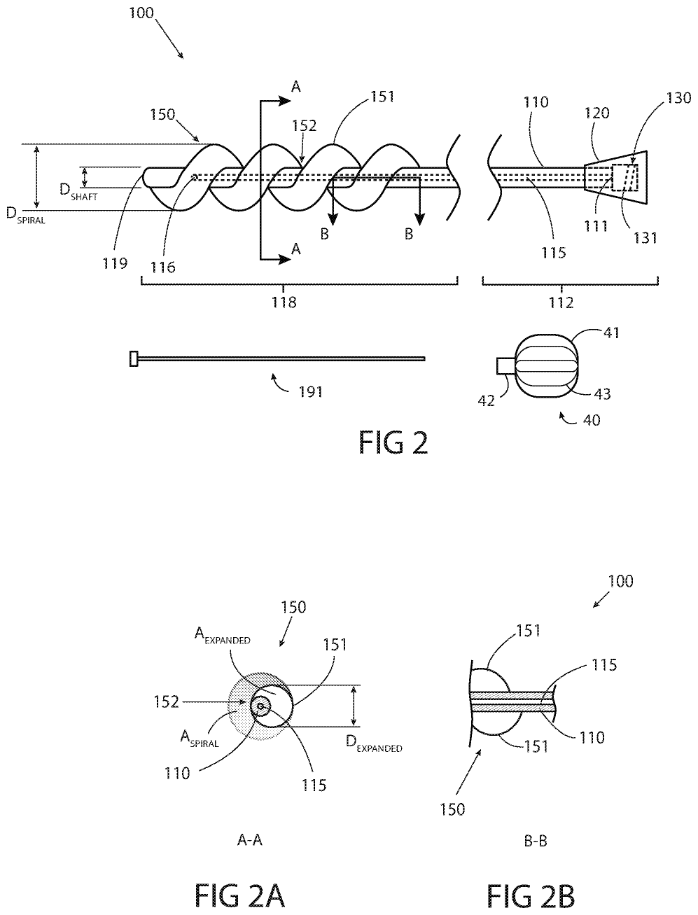

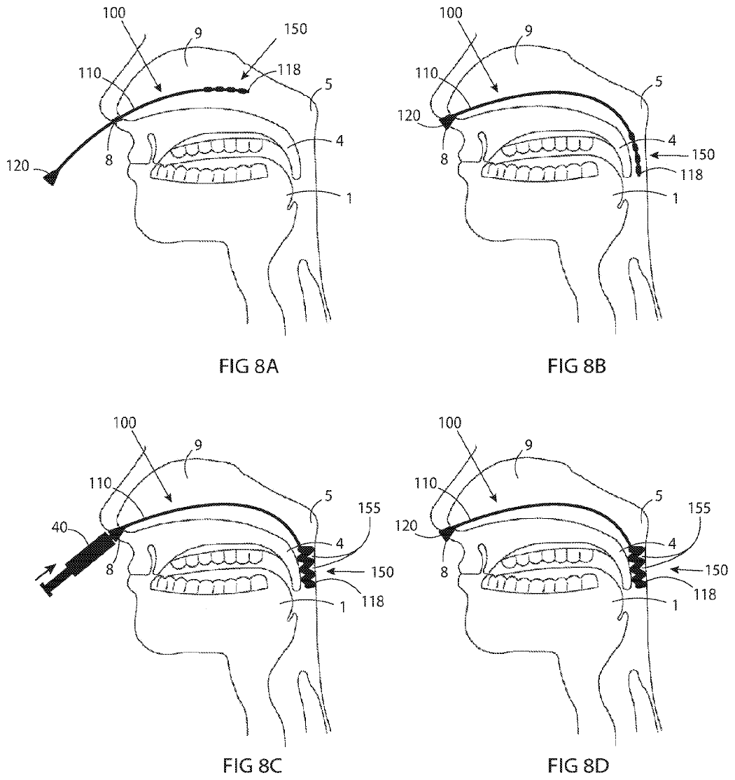

Referring to FIG. 2, a side view of an airway scaffolding device, in an expanded condition, is illustrated, including a scaffolding element shown in a helically expanded state, consistent with the present inventive concepts. Scaffolding device 100 includes an elongate shaft, shaft 110, comprising an outer diameter D_{SHAFT}, a proximal portion 112 including proximal end 111, and a distal portion 118 including distal end 119. A nasal dilator, nosepiece 120 is positioned at or at least proximate the proximal end 111 of shaft 110. Nosepiece 120 is configured to frictionally engage and/or apply a radial force upon the nostril and/or nasal valve 8 of the patient, and can be positioned such that its proximal end is flush with the nostril of the patient. Nosepiece 120 can comprise relatively rigid materials, relatively flexible materials, or both. In some embodiments, nosepiece 120 can be used to dilate nasal passageway 9 and/or nasal valve 8 of the patient. Nosepiece 120 can be configured to collapse, such as a collapse that occurs by the patient squeezing nosepiece 120, such as to ease in removal of scaffolding device 100 after use. An inflatable scaffolding element of the present inventive concepts, balloon 150, is positioned on the distal portion 118 of shaft 110. Balloon 150 can comprise an expandable element configured to inflate to radially expand into the helical geometry shown in FIG. 2 (e.g. when inflated balloon 150 provides alternating peaks 151 and valleys 152, as shown). When balloon 150 is positioned in an airway of the patient, valleys 152 provide a helical breathing pathway as described herein and shown in FIGS. 8C-D.

Shaft 110 comprises an inflation lumen, lumen 115, which fluidly connects to balloon 150 via opening 116. Lumen 115 extends proximally within shaft 110 as shown. The length of shaft 110 and the positioning of balloon 150 on shaft 110 relative to nosepiece 120 determines the position of balloon 150 relative to the patient's airway tissue.

Shaft 110 and balloon 150 are configured to be inserted into and through a nostril of the patient (e.g. either nostril of the patient), such that balloon 150 can be positioned along all or a portion of soft palate 4. Alternatively or additionally, balloon 150 can be positioned at the base of the patient's tongue 1. Insertion and inflation of balloon 150 can open or maintain the opening of the air passageway disposed behind soft palate 4 and/or tongue 1, such as described herebelow in reference to FIGS. 8A-D.

Shaft 110, balloon 150 and/or at least the distal portion of scaffolding device 100 form a relatively smooth surface (when balloon 150 is in a deflated state), and can include a lubricious coating and/or a friction-reduced surface to improve the comfort to the patient during insertion and/or removal of scaffolding device 100 into and/or from the patient's airway. In some embodiments, shaft 110 and/or balloon 150 comprise a material and/or coating selected from the group consisting of: a hydrophilic coating and/or material; a hydrophobic coating and/or material; a lubricant coating such as a silicone lubricant; polytetrafluoroethylene (PTFE) coating and/or material (e.g. an outer liner); and combinations of one or more of these. Distal end 119 of shaft 110 can comprise a rounded, sealed end, also configured to aid in comfort during insertion.

Scaffolding device 100 further includes a valve assembly, valve 130, which is fluidly attached to lumen 115, such that fluids can be introduced through valve 130 and into balloon 150. Valve 130 can be positioned on the proximal end of shaft 110 as shown in FIG. 2, or positioned more distally, such as within a mid-portion of lumen 115 or a lumen 115 location proximate balloon 150. In some embodiments, valve 130 is integral to nosepiece 120. Valve 130 is configured to maintain fluid within balloon 150 (e.g. prevent or at least reduce egress of fluid from balloon 150) after inflation of balloon 150, such as to maintain fluid within balloon 150 at a sufficient pressure to properly provide a scaffolding force to the soft palate 4 and/or other airway tissue of the patient. In some embodiments, valve 130 comprises a one-way valve, such as a valve configured to allow fluid to enter lumen 115 via valve 130, but not exit lumen 115 via valve 130 (e.g. until valve 130 is opened, removed, or otherwise deactivated as described below). In some embodiments, valve 130 is configured to maintain a pressure within balloon 150, such as to maintain pressure at a level of at least 5 psi, at least 10 psi, at least 30 psi or at least 50 psi. In some embodiments, valve 130 can be configured as a pressure-relief valve, such that if fluid in lumen 115 or balloon 150 exceeded a desired level, valve 130 would allow fluid to evacuate device 100. In some embodiments, valve 130 comprises a cracking pressure (e.g. a minimum pressure to introduce fluid into lumen 115) of at least 0.1 psi, or at least 0.5 psi. In some embodiments, valve 130 comprises a cracking pressure of no more than 50 psi, or no more than 200 psi.

Valve 130 can be further configured to be opened, such as to allow deflation of balloon 150 (e.g. to allow fluid to evacuate balloon 150) after a night's sleep and prior to removal of scaffolding device 100 from the patient. Alternatively, scaffolding device 100 can be removed from the patient with balloon 150 inflated, or at least partially inflated. In some embodiments, valve 130 is configured to be opened via compression of valve 130 (e.g. compression applied by squeezing the patient's nose which results in a compressive force applied to valve 130), to allow balloon 150 to deflate and/or fluid to be introduced through valve 130. Alternatively or additionally, valve 130 can be configured to be removed (e.g. detached from shaft 110 and/or lumen 115), such that balloon 150 can deflate.

Valve 130 can comprise a valve selected from the group consisting of: duck-bill valve; slit valve; spring-activated valve; electronically actuatable valve; one-way valve; and combinations of one or more of these. In some embodiments, valve 130 comprises a one-way valve or other valve configured to allow fluid to pass into scaffolding device 100 when a provided fluid exceeds a particular delivery pressure (e.g. exceeds a cracking pressure).

One or more components of scaffolding device 100 can be attached to one or more other components of scaffolding device 100 via one or more adhesives, such as cyanoacrylate, an ultraviolet (UV) light curable adhesive, and/or other adhesive (e.g. adhesive 35 described herebelow). For example, nosepiece 120 can be attached to shaft 110 and/or valve 130 with an adhesive; valve 130 can be attached to shaft 110 with an adhesive; and/or balloon 150 or other scaffolding element of the present inventive concepts can be attached to shaft 110 with an adhesive.

In some embodiments, scaffolding device 100 further includes a fluid delivery assembly, fill assembly 40, which can be fluidly attached (e.g. pre-attached and/or attachable) to valve 130 (e.g. via threads 131 of valve 130) and/or otherwise fluidly attached to lumen 115 (e.g. via threads on the end of shaft 110). In some embodiments, fill assembly 40 comprises a simple syringe, such as a syringe comprising a luer configured to attach to a mating luer of scaffolding device 100 (e.g. threads 131 comprising male or female mating luer threads for attachment to the syringe luer connector). Alternatively, fill assembly 40 can comprise a flexible pouch, such as fill chamber 41 shown, with a connector, connector 42, that fluidly attaches to threads 131 of valve 130 and/or a mating connector attached to lumen 115. Fill chamber 41 is filled (pre-filled or Tillable by the patient) with one or more liquids or gases, such as room air. Fill chamber 41 can include one or more resilient ribs, ribs 43 shown, which can be resiliently biased to maintain fill chamber 41 in an expanded state, such as to cause fill chamber 41 to automatically fill with room air when fill chamber 41 is unattached.

When a compressing force is applied to fill chamber 41, such as by the patient squeezing fill chamber 41, the liquids and/or gases pass through valve 130 and lumen 115 into balloon 150, such that balloon 150 expands, such as to the expanded state shown in FIG. 2. Valve 130 is configured such that when fill assembly 40 is removed from valve 130 (or otherwise detached from lumen 115), balloon 150 remains in an expanded state (e.g. no or minimal fluid egress from balloon 150 occurs). When valve 130 is deactivated or removed, as described hereabove, balloon 150 can deflate, with the expansion fluid exiting via inflation lumen 115.

In some embodiments, lumen 115 is configured to slidingly receive a filament, filament 191 shown, such as a filament configured to be temporarily inserted within scaffolding device 100 (e.g. inserted within lumen 115) to temporarily increase the stiffness of scaffolding device 100 to assist in insertion of balloon 150 into the patient's airway to a location proximate soft palate 4. In some embodiments, scaffolding device 100 includes a second nosepiece 120, not shown but such as a second component for placement into the patient's other nasal valve 8 (e.g. to reduce collapse of that nasal valve).

Referring additionally to FIGS. 2A and 2B, sectional and side sectional views of the airway scaffolding device 100 of FIG. 2 are illustrated, respectively, consistent with the present inventive concepts. FIG. 2A is a cross section at A-A of FIG. 2, and FIG. 2B is a cross section at B-B of FIG. 2. Balloon 150 is expanded to a diameter, D_{EXPANDED} shown, which correlates to an area, A_{EXPANDED} also shown. As shown in FIG. 2A, at one or more points (e.g. all or at least a majority of points) along the length of balloon 150, the cross sectional area (A_{EXPANDED} shown, respectively) occupied by an expanded balloon 150 and shaft 110, at that location, can be less than the overall cross sectional area (at that same location, cross sectional area A_{SPIRAL} shown) of a cylinder circumscribed about the length of balloon 150. As shown in FIG. 2, the diameter of the circumscribed cylinder comprises a diameter D_{SPIRAL}. The areas of A_{SPIRAL} not occupied by the corresponding areas A_{EXPANDED}, at each point along the length of balloon 150, collectively define a pathway that provides for the passage of air along the length of balloon 150, referred herein as the “helical breathing pathway”. The major diameter of the spiral defined by the helical breathing pathway also approximates D_{SPIRAL} (e.g. minimal tissue protrudes into the valleys 152 of expanded balloon 150 such that the periphery of the helical breathing pathway approximates the edge of the above circumscribed cylinder).

Shaft 110 can comprise a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations of one, two or more of these. Alternatively or additionally, shaft 110 can comprise one or more metals (e.g. in a guidewire-like construction or other coiled arrangement), such as a metal selected from the group consisting of: stainless steel; nitinol; and combinations of one or two of these. Shaft 110 can comprise a coating, such as described herebelow.

Shaft 110 can comprise an outer diameter, D_{SHAFT} shown, of between 0.018″ and 0.300″, or between 0.030″ and 0.120″. D_{SHAFT} can comprise a relatively constant diameter or a varying diameter along the length of shaft 110. Shaft 110 can comprise an inner diameter (i.e. the diameter of lumen 115) of at least 0.005″ or at least 0.015″. Shaft 110 can comprise a length of at least 4 cm or at least 6 cm, such as a length of between 6 cm and 30 cm, between 8 cm and 20 cm, or between 10 cm and 13 cm.

Scaffolding elements of the present inventive concepts include balloon 150 of FIGS. 2, 2A and 2B, as well as balloon 1501, scaffolding element 1502, scaffolding element 1503, and/or balloon 1504, each described herebelow. It will be understood that, unless explicitly stated otherwise, elements 1501, 1502, 1503, and 1504 are to be considered alternative embodiments of balloon 150. Elements 1501, 1502, 1503, and 1504 can be of similar construction and arrangement to the similar components of balloon 150 as described herein. These scaffolding elements, or components thereof (e.g. tube 154 described herebelow), hereinafter “scaffolding elements”, can comprise compliant materials, non-compliant materials, or both. Scaffolding elements, or portions of scaffolding elements, that are constructed of one or more non-compliant materials can be configured to inflate to a fixed geometry (e.g. fixed diameter, such as a fixed diameter D_{EXPANDED} described herebelow), wherein additional filling has minimal impact on further expansion. Scaffolding elements, or portions of scaffolding elements, that are constructed of one or more compliant materials can be configured to continually expand as additional filling fluid is introduced. These scaffolding elements can comprise a compliant or non-compliant material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations of one, two, or more of these. In some embodiments, one or more portions (e.g. exterior portions) of balloon 150, or other scaffolding element of the present inventive concepts, can comprise a coating, such as is described herebelow.

Balloon 150 and the other scaffolding elements of the present inventive concepts can comprise a length of up to 4 cm, up to 7 cm, or up to 10 cm, such as a length of between 1 cm and 4 cm, or between 1.5 cm and 2.8 cm. Scaffolding elements of the present inventive concepts can comprise a thickness (e.g. a wall thickness prior to inflation) of at least 0.0001″, 0.001″, and/or 0.010″. Scaffolding elements of the present inventive concepts can be positioned such that the distal end of the scaffolding element is within 10 mm of the distal end of the scaffolding device, or within 5 mm of the distal end of the scaffolding device (such as to prevent or at least reduce the likelihood of the tip of scaffolding device 100 triggering a gag reflex in the patient or otherwise causing discomfort). Scaffolding elements of the present inventive concepts can be configured to provide (e.g. when expanded) a spiral with a major diameter (D_{SPIRAL} herein) of between 0.5 cm and 5 cm, between 1.5 cm and 2.5 cm, or between 1.7 cm and 2.0 cm. Scaffolding elements of the present inventive concepts can provide a helical breathing pathway that comprises a cross sectional area at one or more points along its length, that is at least 10%, at least 20%, at least 35%, or at least 45% of the area defined by the major diameter of the scaffolding element (when the scaffolding element is expanded into the helical geometry). For example, for an expanded scaffolding element comprising a spiral with a major diameter of 2.0 cm, which defines an area of approximately 3.14 cm², the provided helical breathing pathway can comprise an air passageway with a cross sectional area of approximately at least 0.314 cm², 0.628 cm², 1.099 cm², or 1.413 cm², respectively, at one or more points along the length of the scaffolding element.

Balloon 150 and the other scaffolding elements of the present inventive concepts can comprise a burst pressure of at least 5 psi, at least 10 psi, at least 30 psi or at least 50 psi. The scaffolding elements of the present inventive concepts can comprise a fill volume (a volume of fluid to be introduced to achieve a full expansion) of at least 1 ml, 3 ml, 5 ml, or 7 ml. The scaffolding elements of the present inventive concepts can comprise a fill volume (a volume of fluid introduced to cause full expansion of balloon 150 or other scaffolding element) that is less than or equal to 30 ml, or less than or equal to 20 ml. The scaffolding elements of the present inventive concepts can comprise a pitch (e.g. distance between peaks 151 or valleys 152) of at least 2 mm, 4 mm, or 6 mm, or a pitch of no more than 20 mm, 15 mm, or 10 mm.

The scaffolding elements of the present inventive concepts can comprise materials, supporting elements, and/or other features, that maximize the volume of the helical breathing pathway, and/or minimize restrictions within the helical breathing pathway, when the scaffolding element is positioned within a patient airway and expanded (e.g. inflated).

In some embodiments, a scaffolding element of the present inventive concepts (e.g. balloon 150 of FIGS. 2, 2A and 2B, as well as balloon 1501, scaffolding element 1502, scaffolding element 1503, and/or balloon 1504 described herebelow), shaft 110, and/or another component of scaffolding device 100 includes a coating, such as a coating selected from the group consisting of: a lubricous coating; an antibiotic; an antihistamine; an analgesic (e.g. an analgesic to provide comfort during insertion of device 100); and combinations of one or more of these.

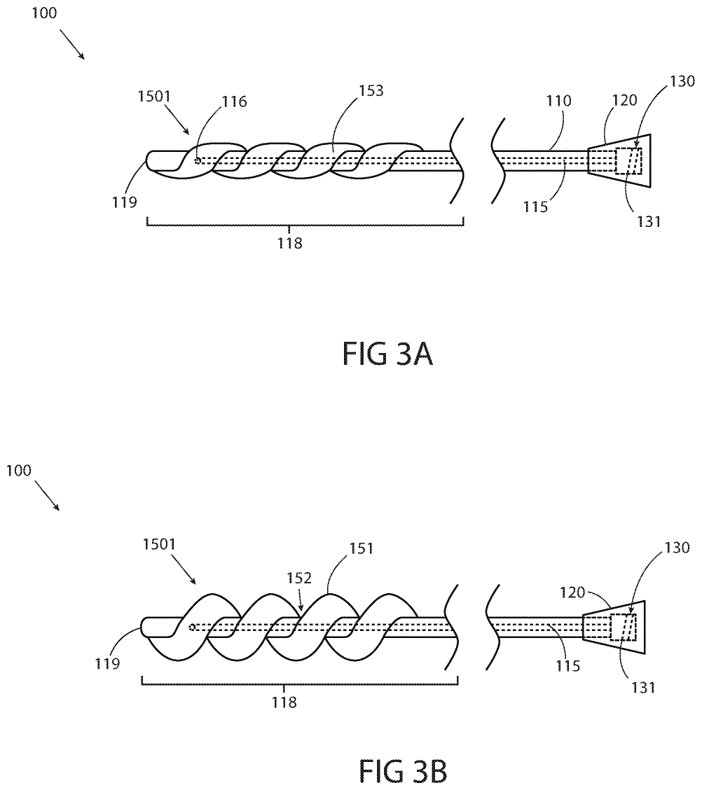

Referring to FIGS. 3A and 3B, side views of an airway scaffolding device, in unexpanded and expanded conditions, respectively, are illustrated, consistent with the present inventive concepts. Scaffolding device 100 includes similar components to scaffolding device 100 described hereabove in reference to FIG. 2, such as shaft 110, nosepiece 120, and valve 130. Scaffolding device 100 of FIGS. 3A-B includes an expandable (e.g. inflatable) scaffolding element, balloon 1501, positioned on the distal portion 118 of shaft 110. Balloon 1501 can comprise a flexible tube, tube 153, that is wrapped around shaft 110 (e.g. wrapped in a helical arrangement) in a manufacturing process of device 100. In some embodiments, tube 153 is fixedly attached along a portion of the length of shaft 110 (e.g. along all or a portion of the contacting surface between tube 153 and shaft 110). In some embodiments, tube 153 is fixedly attached to shaft 110 at two or more discrete locations (e.g. at least fixedly attached to a first location on shaft 110 proximate to distal end 119, and to a second location on shaft 110 more proximal to distal end 119). Distal portion 118 of shaft 110 can include an opening 116 to facilitate the transport of a fluid between lumen 115 and tube 153 (e.g. to deliver fluid to expand tube 153 into the geometry shown in FIG. 3B and/or to remove fluid to allow tube 153 to contract back into the geometry shown in FIG. 3A). In some embodiments, fill assembly 40, not shown but described hereabove in reference to FIG. 2, is attached to lumen 115 (e.g. via valve 130 or otherwise) to deliver a fluid into lumen 115, such that tube 153 is inflated via opening 116. Once inflated, tube 153 can be oriented in a helical geometry that includes alternating peaks 151 and valleys 152, as shown, such as to maintain a helical breathing pathway through the patient's air passageway disposed behind the soft palate 4 and/or tongue 1, such as is described herebelow in reference to FIGS. 8A-D and otherwise herein.

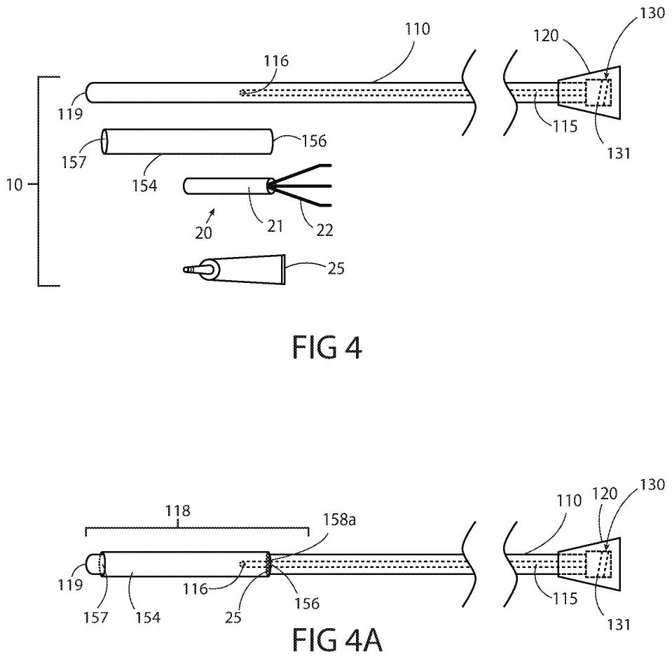

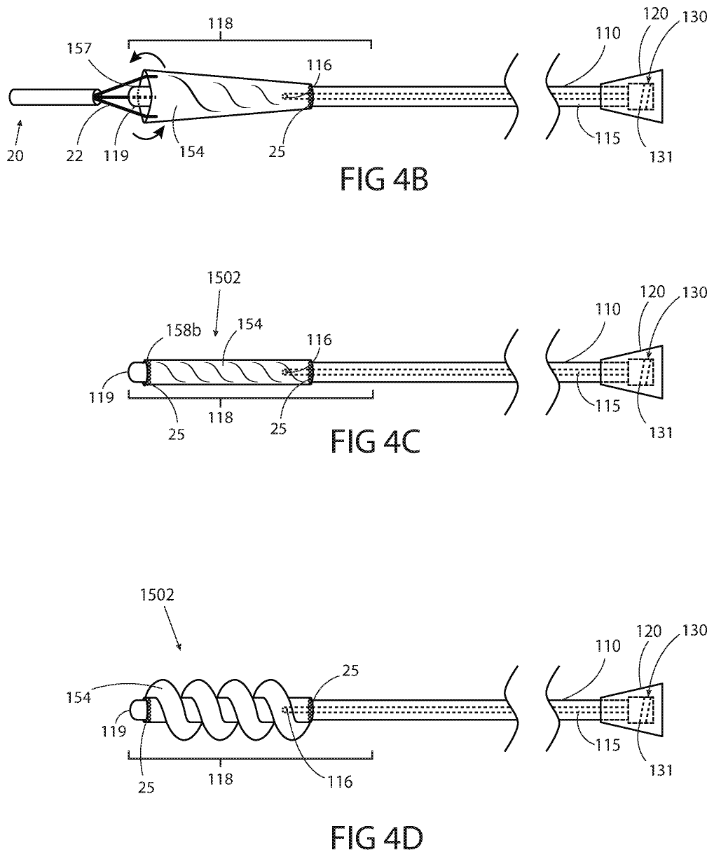

Referring to FIG. 4, a kit for constructing an airway scaffolding device is illustrated, consistent with the present inventive concepts. Kit 10 includes an expandable scaffolding element of the present inventive concepts, tube 154, as well as an adhesive 25, and an expansion tool 20. Kit 10 can further include shaft 110 and nosepiece 120 fixedly attached thereto, as shown. Tube 154 can comprise a hollow tube with proximal end opening 156 and a distal end opening 157. Tube 154 can be expandable or comprise one or more expandable portions. Tube 154 can comprise a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations of one, two or more of these. Kit 10 can further include a valve assembly, valve 130, as described herein. Shaft 110, nosepiece 120, and valve 130, can be of similar construction and arrangement to the similar components described hereabove in reference to FIG. 2. Adhesive 25 can comprise an adhesive material selected from the group consisting of: a curable adhesive (e.g. a UV light or other light curable adhesive); cyanoacrylate; a solvent (e.g. a solvent of tube 154 and/or shaft 110, or both); and combinations of one, two, or more of these. Expansion tool 20 can comprise a handle 21, and two or more filaments 22 attached thereto. Filaments 22 can extend radially from the axis of handle 21, as shown. Distal portion 118 of shaft 110 includes opening 116 as shown to facilitate the transport of fluid between lumen 115 and tube 154, as described herein.

Referring additionally to FIGS. 4A-D, steps for constructing a scaffolding device using kit 10 of FIG. 4 are illustrated, consistent with the present inventive concepts. As shown in FIG. 4A, shaft distal portion 118 can slidingly receive tube 154 via opening 156, such that distal end 119 exits and extends beyond opening 157, and tube 154 circumferentially surrounds shaft 110 within the distal portion 118 of shaft 110. In some embodiments, adhesive 25 is applied (e.g. circumferentially applied) to a first portion of shaft 110 to fixedly attach a first portion of tube 154 to shaft 110 at a fixation point 158a (e.g. a circumferential fixation point). In some embodiments, fixation point 158a is positioned at a proximal portion of tube 154, at or near the proximal end of tube 154, as shown. As shown in FIG. 4B, expansion tool 20 can be inserted into opening 157 of tube 154, such that filaments 22 engage the inner walls of tube 154. Filaments 22 can exert a force on the inner walls of tube 154 to radially expand the distal portion of tube 154. A user can rotate expansion tool 20 to cause a deformation, such as a twist of tube 154 from its proximal end to its distal end. In some embodiments, expansion tool 20 rotates tube 154 between 10° and 2160°, such as a rotation of between 90° and 1080°, or between 360 and 1080. As shown in FIG. 4C, adhesive 25 can be applied (e.g. circumferentially applied) to a second portion of shaft 110 to fixedly attach a second portion of tube 154 to shaft 110 at a fixation point 158b (e.g. while maintaining the twisting described hereabove). In some embodiments, fixation point 158b (e.g. a circumferential fixation point) is positioned in a distal portion of tube 154, at or near the distal end of tube 154, as shown. Fixation point 158b can be configured to secure a portion of tube 154 to shaft 110, such that the twist or other deformation as described hereabove in reference to FIG. 4B, is preserved once filaments 22 disengage (e.g. are withdrawn or otherwise removed) from the inner walls of tube 154. In some embodiments, a circumferential clamp is positioned about fixation point 158a and/or 158b during curing (e.g. UV light curing) of the adhesive 25. Fixation points 158a and 158b provide a seal between tube 154 and shaft 110, such that a liquid that is delivered into tube 154 (e.g. via opening 116) can cause tube 154 to expand and maintain an expanded state. As a result of the manufacturing steps shown in FIGS. 4A-C, an expandable scaffolding element of the present inventive concepts, scaffolding element 1502 is formed, including tube 154 which has been fixedly attached to shaft 110 (e.g. in a twisted arrangement such that tube 154 inflates in the spiral geometry shown in FIG. 4D). Scaffolding element 1502 can be inflated or otherwise expanded via valve 130, lumen 115, and inflation opening 116, such as using fill assembly 40, as described herein. Insertion of scaffolding element 1502 thru the nasal passageway 9 to a location behind the soft palate 4 or tongue 1, and inflation of scaffolding element 1502 (e.g. subsequent inflation), provides a helical breathing pathway of the present inventive concepts, such as is described herebelow in reference to FIGS. 8A-D.

In some embodiments, no or minimal twist is applied to tube 154 (e.g. between the proximal end and distal end of tube 154). Tube 154 can comprise an inner diameter (ID) when in its resiliently biased state (e.g. relaxed state) that is smaller than the outer diameter (OD) of the distal portion 118 of shaft 110 (the resiliently biased diameter of the distal portion 118 of shaft 110), such that tube 154 is in a stretched state after positioned about shaft 110. This stretched condition can be configured to cause tube 154 to expand into a helical geometry during inflation (e.g. obviating the need for the twisting of tube 154 to cause the helical expansion). In some embodiments, the resiliently biased ID of tube 154 is at least 30%, 40%, 50%, 60%, or 70% smaller than the resiliently biased OD of the distal portion 118 of shaft 110 about which tube 154 is positioned.

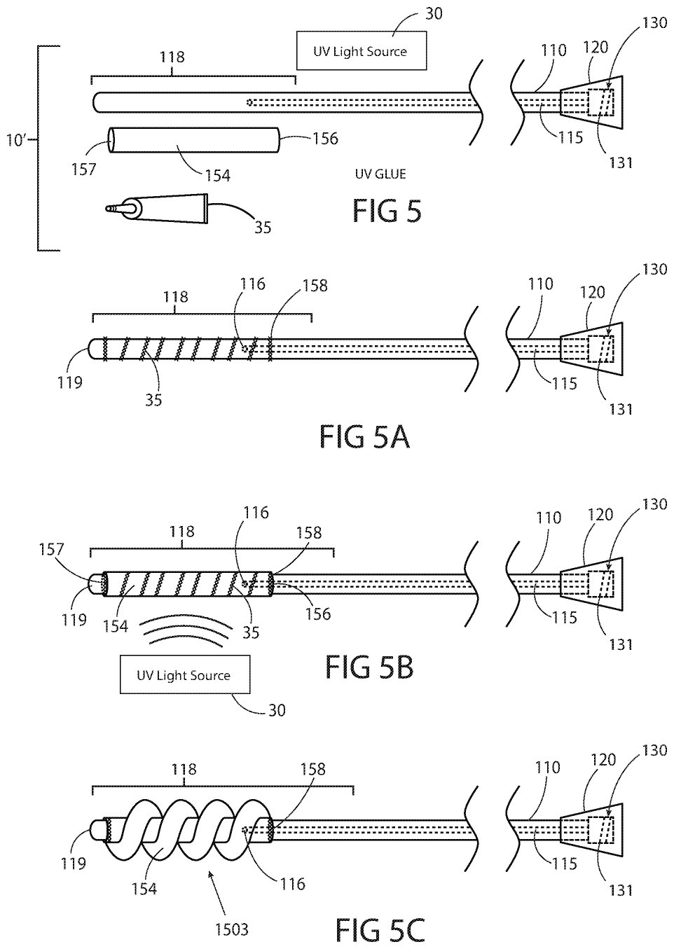

Referring to FIG. 5, a kit for constructing an airway scaffolding device is illustrated, consistent with the present inventive concepts. Kit 10′ includes an expandable element, tube 154, used to create a scaffolding element of the present inventive concepts, tube 154, as well as an adhesive, adhesive 35 (e.g. a UV light curing adhesive), and a curing device 30 (e.g. a UV light source). Kit 10′ can further include shaft 110 with nosepiece 120 fixedly attached thereto, as shown. Kit 10 can further include a valve assembly, valve 130, as described herein. Shaft 110, nosepiece 120, and valve 130, can be of similar construction and arrangement to the similar components described hereabove in reference to FIG. 2. Tube 154 can comprise a hollow tube with a proximal end opening 156 and a distal end opening 157. Tube 154 can be of similar construction and arrangement as tube 154 described hereabove in reference to FIGS. 4 and 4A-D. In some embodiments, distal portion 118 of shaft 110 includes opening 116 as shown to facilitate the movement of a fluid between lumen 115 and tube 154.

Referring additionally to FIGS. 5A-C, steps for constructing a scaffolding device using kit 10′ of FIG. 4 are illustrated, consistent with the present inventive concepts. Kit 10′ includes tube 154, adhesive 35, curing device 30, and shaft 110 with nosepiece 120 fixedly attached thereto. As shown in FIG. 5A, adhesive 35 can be applied to distal portion 118 of shaft 110 to create one or more continuous and/or discrete fixation points 158. In some embodiments, adhesive 35 is applied between the contacting portions of tube 154 and the distal portion 118 of shaft 110 in a pattern (e.g. a helical pattern). In some embodiments, adhesive 35 is applied in a continuous line that wraps or otherwise surrounds distal portion 118 in a helical pattern. As shown in FIG. 5B, shaft distal portion 118 can slidingly receive tube 154 via opening 156, such that distal end 119 exits and extends beyond opening 157. Tube 154 can make contact with fixation points 158. In some embodiments, tube 154 is unrolled, expanded, or otherwise placed about distal portion 118 in a manner in which adhesive 35 is not disturbed and/or the patterned (e.g. helical) placement of adhesive 35 is not disrupted. Additionally or alternatively, adhesive 35 can comprise a relatively hard (pre-cure) material, or other consistence material whose geometry of placement is not significantly affected by the placement of tube 154 over shaft 110 distal portion 118.

Curing device 30 can be used to cure, harden, and/or otherwise activate adhesive 35 to fixedly attach tube 154 to the distal portion 118 of shaft 110 in the helical pattern of the applied adhesive 35. As a result of the manufacturing steps shown in FIGS. 5A-B, an expandable scaffolding element of the present inventive concepts, scaffolding element 1503 is formed, including tube 154 which has been fixedly attached to shaft 110. As shown in FIG. 5C, scaffolding element 1503 can be inflated or otherwise expanded via inflation opening 116, such that the tube 154 expands into a helical geometry due to the cured fixation points 158 preventing tube 154 from inflating in the areas where adhesive 35 was applied. Insertion of scaffolding element 1503 thru the nasal passageway 9 to a location behind the soft palate 4 or tongue 1, and inflation of scaffolding element 1503 (e.g. subsequent inflation), provides a helical breathing pathway of the present inventive concepts, such as is described herebelow in reference to FIGS. 8A-D.

In some embodiments, adhesive 35 is applied between tube 154 and a distal portion 118 of shaft 110 without a particular pattern of application (e.g. not necessarily in a helical pattern), such as when adhesive 35 is applied over all or at least a majority of the contacting surfaces between tube 154 and shaft 110. In these embodiments, curing device 30 can be manipulated to cure only a portion of the previously applied adhesive 35, such as to create a patterned adherence between tube 154 and shaft 110 (e.g. a helically shaped pattern of adherence due to curing of a helical portion of the applied adhesive 35). For example, curing device 30 can deliver a focused curing light (e.g. a UV light used to cure a UV-curing based adhesive 35) toward the contacting portion between shaft 110 and tube 154 (with adhesive 35 previously applied) while curing device 30 is simultaneously translated and rotated about the contacting portion (e.g. by rotating curing device 30, the assembly including tube 154, or both, and simultaneously translating curing device 30, the assembly including tube 154, or both, to create a helically shaped pattern of adherence). After the curing step, the remaining uncured adhesive 35 can be removed, such as during a cleaning process including delivering a solvent (e.g. a solvent of adhesive 35) between the unadhered contacting portions between tube 154 and shaft 110.

While the manufacturing steps described in reference to FIGS. 5A-C include a curing step (e.g. a UV curing step) performed using curing device 30, in alternative embodiments, adhesive 35 is applied to shaft 110, tube 154 is expanded and positioned around shaft 110, and tube 154 is allowed to contract to contact shaft 110 without significantly disturbing the spiral geometry of adhesive 35 about shaft 110. In these embodiments, adhesive 35 can be allowed to self-cure (e.g. without the aid of a curing device), over time, such that scaffolding element 1503 is formed and configured to expand in the helical geometry described hereabove. In these embodiments, adhesive 35 can comprise cyanoacrylate or a solvent (e.g. a solvent of tube 154 and/or shaft 110).

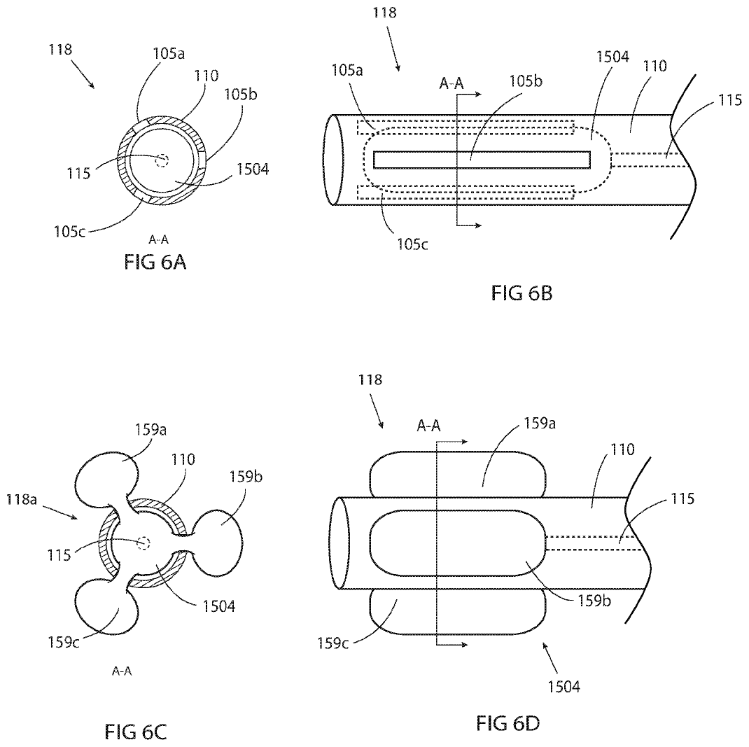

Referring to FIGS. 6A and 6B, cross sectional and side views of the distal portion of an unexpanded balloon in a distal portion of an airway scaffolding device are illustrated, respectively, consistent with the present inventive concepts. Referring additionally to FIGS. 6C and 6D, cross sectional and side views of the balloon of FIGS. 6A-D, inflated to an expanded state are illustrated, also consistent with the present inventive concepts. Scaffolding device 100 comprises shaft 110 with lumen 115 extending from its proximal end, and a valve assembly, valve 130. Scaffolding device 100 can further comprise a nosepiece 120, not shown. Shaft 110, nosepiece 120, and valve 130, can be of similar construction and arrangement to the similar components described hereabove in reference to FIG. 2. As shown in FIGS. 6A and B, an expandable scaffolding element, balloon 1504 is positioned within the distal portion 118 of shaft 110. Balloon 1504 is in fluid communication with lumen 115, such that balloon 1504 can receive a fluid via lumen 115 (e.g. and via valve 130). In some embodiments, distal portion 118 of shaft 110 further includes multiple, for example three or more, elongate openings, slots 105a-c. Balloon 1504 can comprise multiple bulbous portions, lobes 159a-c. When balloon 1504 receives a fluid via lumen 115 (e.g. when balloon 1504 is inflated), lobes 159a-c can pass thru slots 105a-c, respectively, such that lobes 159a-c protrude through slots 105a-c and expand into the geometry shown in FIG. 6C-D. Insertion of balloon 1504 thru the nasal passageway 9 to a location behind the soft palate 4 or tongue 1, and inflation of balloon 1504 (e.g. subsequent inflation), provides a helical breathing pathway of the present inventive concepts, such as is described herebelow in reference to FIGS. 8A-D.

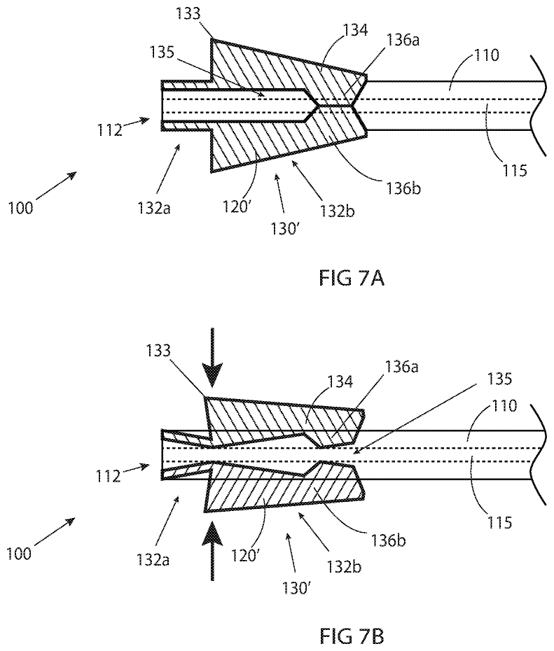

Referring to FIGS. 7A and 7B, side sectional views of the proximal portion of an airway scaffolding device comprising a valve including a nosepiece are illustrated, consistent with the present inventive concepts. Scaffolding device 100 can be of similar construction and arrangement to scaffolding device 100 described hereabove. Scaffolding device 100 of FIGS. 7A-B comprises shaft 110 with lumen 115 extending from its proximal end. Shaft 110 comprises proximal portion 112 that includes a valve assembly, valve 130′, that includes a nasal dilator, nosepiece 120′. Valve 130′ can be configured to transition from a closed condition in which fluid flow through valve 130′ is prevented or at least limited, to an open condition in which fluid can freely flow through valve 130′. Valve 130′ can comprise a first portion 132a and a second portion 132b, with a lumen 135 therethrough, each as shown. Lumen 135 is fluidly attached to lumen 115 of shaft 110 (e.g. which is in fluid communication with a balloon 150 or other expandable element of the present inventive concepts). Second portion 132b can comprise a proximal portion 133 that tapers into a distal portion 134. Distal portion 134 can comprise two or more clamping elements, projections 136a,b, positioned opposite each other and extending into lumen 135. As shown in FIG. 7A, valve 130′ is in a closed condition with projections 136a,b in contact with each other. When in contact, projections 136a,b can be shaped or otherwise configured to obstruct (e.g. prevent or otherwise inhibit) the flow of fluid through lumen 135, and therefore attached lumen 115. Proximal portion 133, projections 136a,b and/or other components of valve 130′ can be resiliently biased in the geometry shown in FIG. 7A. As shown in FIG. 7B, valve 130′ can transition from a closed condition to an open condition when a compression force is applied (e.g. via fingers of the patient) to proximal portion 133. Compression force applied to proximal portion 133 can cause distal portion 134 to extend (e.g. pivot) radially and projections 136a,b to separate. The separation of projections 136a,b can allow for the flow of fluid through lumen 135, and therefore through attached lumen 115. In operation, valve 130′ can be opened to fill and/or empty a scaffolding element of the present inventive concepts (e.g. elements 150, 1501, 1502, 1503, or 1504 as described herein). Valve 130′ can be closed (e.g. resiliently biased closed), to prevent a scaffolding element from expanding (e.g. prior to use), and/or to maintain a scaffolding element in an expanded condition (e.g. during sleep to maintain a helical breathing pathway of the present inventive concepts).

In some embodiments, valve 130′ is configured to maintain fluid within balloon 150 or another expandable scaffolding element of the present inventive concepts at a pressure as described hereabove in reference to valve 130 of FIG. 2.

In some embodiments, valve 130′ is configured to allow fluid to pass into scaffolding device 100 without valve 130′ having to be compressed. For example, an inflation device (e.g. fill assembly 40 described hereabove) can be attached to valve 130′ and sufficient pressure of fluid delivery achieved to cause projections 136a-b to separate and the fluid to pass into lumen 115.

Referring to FIGS. 8A-D, a method of inserting an airway scaffolding device into a patient's airway is illustrated, consistent with the present inventive concepts. Scaffolding device 100 can directly stent or otherwise apply a force to soft palate 4 and/or tongue 1 of a sleep apnea patient for the purpose of maintaining a breathing airway while minimizing any discomfort and allowing for swallowing. As shown in FIG. 8A, patient P inserts distal portion 118 of shaft 110, including a scaffolding element, such as balloon 150 shown, into a nostril. Patient P advances shaft 110 through nasal valve 8 and into nasal passageway 9. As shown in FIG. 8B, patient P further advances shaft 110 through nasal passageway 9 until balloon 150 is proximate to patient P's soft palate 4 and/or base of tongue 1 (e.g. until nosepiece 120 is flush with the nostril of patient P). As shown in FIG. 8C, patient P inflates balloon 150 via a fill assembly 40. Fill assembly 40 can be configured to engage nosepiece 120 (and/or valve 130 described herein) and can introduce a fluid through lumen 115 of shaft 110 and into balloon 150. As shown in FIG. 8D, when inflated, balloon 150 has a helical geometry and applies a scaffolding force to a portion of patient P's airway (e.g. applies a force to soft palate 4) while maintaining helical breathing pathway 155 shown.

In some embodiments, scaffolding device 100 is inserted prior to sleep, and removed and disposed of after use, often referred to as a “single use device”. Alternatively, scaffolding device 100 can be used for multiple sleeping periods (e.g. multiple nightly uses), such as when scaffolding device 100 is cleaned prior to a second insertion into the patient. Cleaning of scaffolding device 100 can be performed, for example, with soap and water, or with alcohol (e.g. isopropyl alcohol). In some embodiments, scaffolding device 100 is configured to be single use, multiple use, or both (e.g. at the patient's discretion).

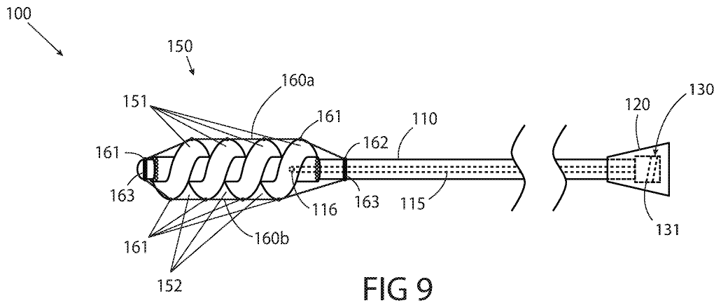

Referring to FIG. 9, a side view of an airway scaffolding device, in an expanded condition, is illustrated, consistent with the present inventive concepts. Scaffolding device 100 can be of similar construction and arrangement to scaffolding device 100 described herein, such as in reference to FIGS. 2, 3, and/or 6. Scaffolding device 100 comprises shaft 110 with lumen 115 extending from its proximal end. Lumen 115 is fluidly connected to a valve assembly, valve 130. Scaffolding device 100 further comprises a scaffolding element, balloon 150. Balloon 150 can comprise one or more filaments 160, such as the two filaments 160a,b shown. Filaments 160a,b can be fixedly attached to balloon 150 via two or more fixation points 161. Fixation points 161 can comprise an adhesive (e.g. a curable adhesive as described herein) applied to peaks 151 of balloon 150 (e.g. to a series of sequential peaks at the same radial position along balloon 150). Filaments 160a,b can be configured to prevent or at least reduce deflection and/or to maintain a uniform deflection of each peak 151 of balloon 150, such as to avoid undesired deflections when a portion of balloon 150 (e.g. one or more of peaks 151) receives an external force (e.g. as applied by a portion of the patient's airway during inflation, positioning, breathing, and/or swallowing). Minimal deflection, or uniform deflection, will maintain sufficient space in each valley 152, such as to provide a minimum helical breathing pathway for the patient (e.g. helical breathing pathway 155 shown in FIGS. 8C-D). In other words, one, two, three or more filaments 160 can be configured to maximize the volume of the helical breathing pathway, and/or minimize restrictions within the helical breathing pathway, when balloon 150 is positioned within a patient airway and expanded (e.g. inflated). In some embodiments, the distal and proximal ends of one or more filaments 160 are attached (e.g. adhesively attached and/or attached via a circumferential band, band 163) to shaft 110, in distal location 161 and proximal location 162, respectively and as shown in FIG. 9.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. An airway scaffolding device for a patient comprising:
an elongate shaft with a distal portion;
a scaffolding element positioned about the distal portion of the elongate shaft, the scaffolding element configured to receive a volume of fluid to expand the scaffolding element to exert a force upon a soft palate and/or base of a tongue of the patient;
a valve assembly positioned proximal to the scaffolding element; and
a lumen disposed between the valve assembly and the scaffolding element, wherein the fluid is introduced through the valve assembly and travels through the lumen into the scaffolding element;
wherein the scaffolding element provides a helical breathing pathway when inserted into an airway of the patient and transitioned to an expanded state via the fluid introduced through the valve assembly; and
wherein the valve assembly is configured to reduce egress of fluid from the scaffolding element to maintain the scaffolding element in the expanded state.

2. The scaffolding device as claimed in at least one of the preceding claims, wherein the device comprises a single-use device.

3. The scaffolding device as claimed in at least one of the preceding claims, wherein the device comprises a multiple-use device.

4. The scaffolding device according to claim 3, wherein the device is configured to be cleaned between uses.

5. The scaffolding device as claimed in at least one of the preceding claims, wherein the scaffolding element is configured to be expanded after insertion into the patient's airway.

6. The scaffolding device as claimed in at least one of the preceding claims, wherein the scaffolding element comprises a balloon.

7. The scaffolding device as claimed in at least one of the preceding claims, wherein the scaffolding element is configured to expand into a helical geometry.

8. The scaffolding device as claimed in at least one of the preceding claims, wherein the scaffolding element is configured to be deflated prior to removal from the patient.

9. The scaffolding device as claimed in at least one of the preceding claims, wherein the scaffolding element is configured to be removed from the patient without deflation.

10. The scaffolding device as claimed in at least one of the preceding claims, wherein the scaffolding element is configured to be filled with a liquid.

11. The scaffolding device as claimed in at least one of the preceding claims, wherein the scaffolding element is configured to be filled with a gas.

12. The scaffolding device according to claim 11, wherein the gas comprises air.

13. The scaffolding device as claimed in at least one of the preceding claims, wherein the scaffolding element comprises a compliant material.

14. The scaffolding device according to claim 13, wherein the scaffolding element further comprises a non-compliant material.

15. The scaffolding device as claimed in at least one of the preceding claims, wherein the scaffolding element comprises a non-compliant material.

16. The scaffolding device as claimed in at least one of the preceding claims, wherein the scaffolding element comprises a material selected from the group consisting of: silicon; nylon; polyethylene; polyurethane; low-durometer polyurethane; high-durometer polyurethane; polytetrafluoroethylene (PTFE); expandable polytetrafluoroethylene (ePTFE); polyethylene terephthalate (PET); polyimide; polyether block amide; latex; and combinations thereof.

17. The scaffolding device as claimed in at least one of the preceding claims, wherein the scaffolding element comprises a length less than or equal to 4 cm.

18. The scaffolding device according to claim 17, wherein the scaffolding element comprises a length less than or equal to 7 cm, and/or less than or equal to 10 cm.

19. The scaffolding device as claimed in at least one of the preceding claims, wherein the scaffolding element comprises a length between 1 cm and 4 cm.

20. The scaffolding device according to claim 19, wherein the scaffolding element comprises a length between 1.5 cm and 2.8 cm.

21.-107. (canceled)
